(19) United States
(12) Patent Application Publication
(10) Pub. No.: US 2026/0127900 A1
(43) Pub. Date: May 7, 2026

(54) ACTIVE PROMPT TUNING OF VISION-LANGUAGE MODELS FOR HUMAN-CONFIRMABLE DIAGNOSIS AND EXPLANATION OF MICROSCOPY IMAGES

(71) Applicant: UNIVERSITY OF SOUTH FLORIDA, Tampa, FL (US)

(72) Inventors: Lawrence Hall, Tampa, FL (US); Dmitry Goldgof, Tampa, FL (US); Abhiram Kandiyana, Tampa, FL (US); Peter Mouton, Saint Petersburg, FL (US)

(21) Appl. No.: 19/226,117

(22) Filed: Jun. 2, 2025

Related U.S. Application Data
(60) Provisional application No. 63/715,252, filed on Nov. 1, 2024.

(51) Int. Cl. G06V 20/69; G06V 10/778; G16H 30/40; G16H 50/20

(52) U.S. Cl. CPC G06V 20/698; G06V 10/7788; G06V 20/693; G16H 30/40; G16H 50/20

(57) ABSTRACT

Systems and methods are provided herein for developing and deploying active-prompt-tuned applications for automated diagnosis and categorization of microscopy slide images. Processes of the present disclosure may control and provide for human-confirmable diagnostic classifications of such slide images, based on controlled instructions provided to vision-language models. The controlled instructions may be developed by systems provided herein, which generate system prompts and example prompt sets using active prompt tuning approaches.

### CROSS REFERENCE TO RELATED APPLICATIONS

This application claims priority to U.S. provisional patent application Nos. 63/654,302, filed May 31, 2024, and 63/715,252, filed Nov. 1, 2024, the entire contents of which are incorporated herein by reference.

### STATEMENT OF GOVERNMENT SUPPORT

This invention was made with government support under grant numbers 1513126, 1746511, and 1926990 awarded by the National Science Foundation. The government has certain rights in the invention.

### BACKGROUND

Accurate diagnosis of real-world problems based on visual perception and visual-based reasoning-whether from first-person viewing, images, or video—is an important requirement across numerous domains, including medical diagnostics, industrial inspection, environmental monitoring, food and agricultural inspection, and scientific research (to name just a few). Having a human expert personally examine the objects, organisms, etc. to diagnose problems is also not feasible: humans simply cannot see many of the visual traits important to diagnosis (e.g., because they are too small, too numerous, too fast, etc.). Thus, inclusion of computer assistance in visual diagnosis has become a common approach in some fields.

Recent approaches to computer-assisted visual diagnoses have relied on supervised deep learning models, such as convolutional neural networks (CNNs), which require extensive labeled datasets and significant computational resources. These methods often involve time-consuming processes for data annotation, model selection, hyperparameter tuning, and iterative training cycles. Moreover, the need for domain-specific expertise to generate accurate ground truth labels presents a bottleneck in scalability and reproducibility, and can redirect experts' time away from current diagnostic tasks.

In medical imaging, for example, rendering diagnoses of disease conditions based on imaging usually requires expert (radiologist, pathologist, etc.) review and annotation of images (e.g., adding annotations to MRI studies; identifying and annotating cellular features in microscopy images of tissue sections, etc.). While CNN-based models have relatively high accuracy in such tasks, they are limited by their dependence on large, annotated datasets and the need for retraining when applied to new imaging modalities, magnifications, or biological targets.

Recent advances in vision-language models (VLMs) could, in theory, offer a promising alternative. These models are capable of interpreting and reasoning over both visual and textual inputs, enabling them to perform classification tasks of images.

As shown in line 502 of FIG. 2, initial VLMs generally require millions of image-caption pairs for training (meaning they are almost all general-purpose at the start), which itself can be incredibly expense and resource intensive (e.g., several millions of dollars and 10+ days to train or retrain certain VLMs). As shown in line 504, for the VLM to be trained to a given task or subject matter still would require around 50,000 image-caption pairs and significant cost, and still may not reliably provide diagnoses. Due to absence of readily-available, public datasets, attempts at fine-tuning VLMs have not been feasible or widespread (in part, given the cost and constant updating of VLM base models).

Thus, some attempts at improving behavior and accuracy of general VLMs have involved using a few representative examples of the type of classification that is desired, and providing them at inference time—a technique known as few-shot prompting. Such approaches can reduce the need for extensive training and domain-specific fine-tuning, but can result in inconsistent outputs, rely on general VLMs that are subject to modification and retraining by their owners (e.g., Google, OpenAI, etc.) and still require expert development of the few examples. And, users generally are not able to interpret results or ascertain why/whether a given output occurred (e.g., if incorrect or unexpected).

Accordingly, a need exists for a feasible and reliable approach to leveraging the power of VLMs for highly-accurate, domain-specific visual diagnostic assistance. Such approach should be able to utilize the strength of very high parameter models, but avoid a need for large-scale retraining or fine tuning. Additionally, the approach should ensure consistency and reliable output, avoiding background changes to model weights or structure implemented by developers/owners of the models. Finally, the approach should be capable of easy refinement, customization, and personalization within a given domain-specific diagnostic task, without needing a large amount of new training data.

### SUMMARY

The following presents a simplified summary of the disclosed technology herein in order to provide a basic understanding of some aspects of the disclosed technology. This summary is not an extensive overview of the disclosed technology. It is intended neither to identify key or critical elements of the disclosed technology nor to delineate the scope of the disclosed technology. Its sole purpose is to present some concepts of the disclosed technology in a simplified form as a prelude to the more detailed description that is presented later.

In some aspects, the present disclosure can provide methods for human-confirmable diagnostic classification and explanation of microscopy images, leveraging the advantages and techniques described herein. Such methods may relate to development and/or deployment of active-prompt-tuned, microscopy-study specific classification applications. For example, a method may comprise: receiving criteria information for a given class of microscopy study, the criteria information comprising: a set of possible slide classification labels, descriptions of visual features of anatomy anticipated to be found in microscopy slide images for each of the slide classification labels, and slide image preparation information describing: a sample acquisition method used to prepare biological samples appearing in the slides; a stain method used to stain structures within the samples; and a magnification used to acquire images of the samples; receiving a set of unclassified slide images prepared using the sample acquisition method, the stain method, and the magnification; creating an Active Set of unclassified images by random sampling of the set of unclassified images; generating a System Prompt based on the criteria information and input/output characteristics of a diagnostic review platform for microscopy images; presenting an Initial Prompt subset of the Active Set of images to a human reviewer, and requiring the human reviewer to select one of the image classification labels and provide an unstructured feature explanation, for each image of the Initial Prompt subset; providing images of the Active Set as input to a vision-language model (VLM) with an instruction comprising the System Prompt and a Prompt Set comprising the Initial Prompt subset; iteratively displaying to the human reviewer candidate image-caption pairs comprising the images of the Active Set together with associated predicted labels and predicted unstructured feature explanations derived from VLM outputs; requiring the human reviewer to review the image-caption pairs and confirm, reject, or edit them; for each image-caption pair approved or edited by the human reviewer, adding them to the Prompt Set; generating a diagnostic profile specific to the given class of microscopy study based on the System Prompt and Prompt Set; and storing the diagnostic profile in a memory associated with the diagnostic review platform for use in automatically generating image classifications and supporting feature explanations for future slide images of the given class of microscopy study.

In another aspect the present disclosure may provide for systems configured to develop and/or deploy active-prompt-tuned diagnostic applications for particular classes of microscopy study. Such systems may comprise a processor, a network interface, and a memory having stored thereon software instructions which, when executed, cause the system to perform a method such as the foregoing. Such systems may further store libraries of active prompt tuned diagnostic profiles, and utilize them to manage interactions with VLMs in diagnostic image review tasks.

### BRIEF DESCRIPTION OF THE DRAWINGS

The foregoing features of embodiments will be more readily understood by reference to the following detailed description, taken with reference to the accompanying drawings, in which:

FIG. 1 is a flowchart illustrating an example process for active-prompt tuning for image-based diagnostic tasks, according to some embodiments.

FIG. 2 is a block diagram depicting various configurations and data flow paths among hardware resources in systems according to some embodiments.

FIG. 3 is a block diagram showing data composition and software management of VLM interactions for a microscopy task according to some embodiments.

FIG. 4 is a block diagram showing data composition and software management of VLM interactions as well as iterative refinement processes for a visual fruit examination task according to some embodiments.

FIG. 5 is a comparative flowchart showing various approaches to VLM generation and adaptation.

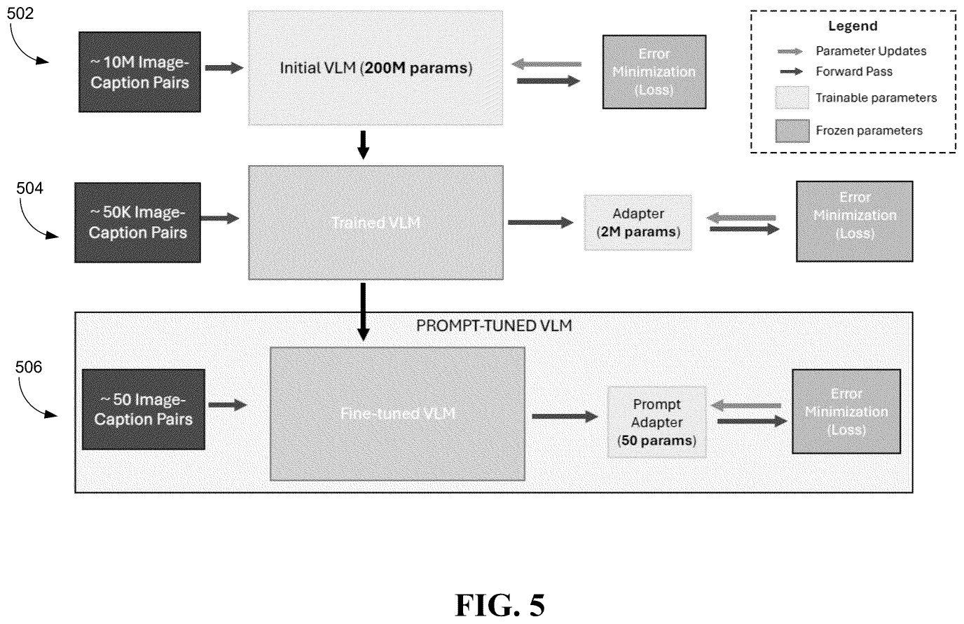

### DETAILED DESCRIPTION

The following description will provide a disclosure of various features, approaches, and aspects of example systems and methods that can overcome the limitations described above, and allow for more rapid generation of model-interpretable ground truth examples by human experts, improved human insight into model behavior, reliable consistency and predictability of model behavior, and elimination of time and resources usually entailed in training or fine-tuning a model while still achieving the tailored behavior of a trained/tuned model. First, a general description will be provided of aspects of technologies that may be utilized in systems and methods of the present disclosure. Second, an overview of illustrative system/hardware architectures will be provided along with an overview of a framework for deploying certain processes and algorithms of the present disclosure. Third, a description of the inventors' experiments and validation studies will be provided.

Described here are systems and methods directed to a human-in-the loop, active prompt tuning approach to refining behavior of vision-language models to produce improved outputs. The approaches described herein use algorithms for generating and for iteratively verifying confirmed ground truth image/label pairs, but in a novel way that leverages both structured and unstructured information about the pertinent content of each image that is (to a human expert) diagnostically relevant to the domain-specific diagnosis task. The proposed algorithms can also simultaneously leverage textual domain specification information from recognized/standardized resources (like diagnosis protocols) in addition to specific, unique human expert input, in a variety of novel ways.

The present disclosure will now provide overview descriptions of various approaches to deploying embodiments of such systems and methods. It should be understood that the processes and algorithms described below are not limiting of the scope of this disclosure, can be combined in various configurations, and may be adapted to replace, complement, and/or fit with existing platforms for image-based diagnoses.

### Example Process for Developing Domain/Diagnosis-Specific Model Interaction Framework

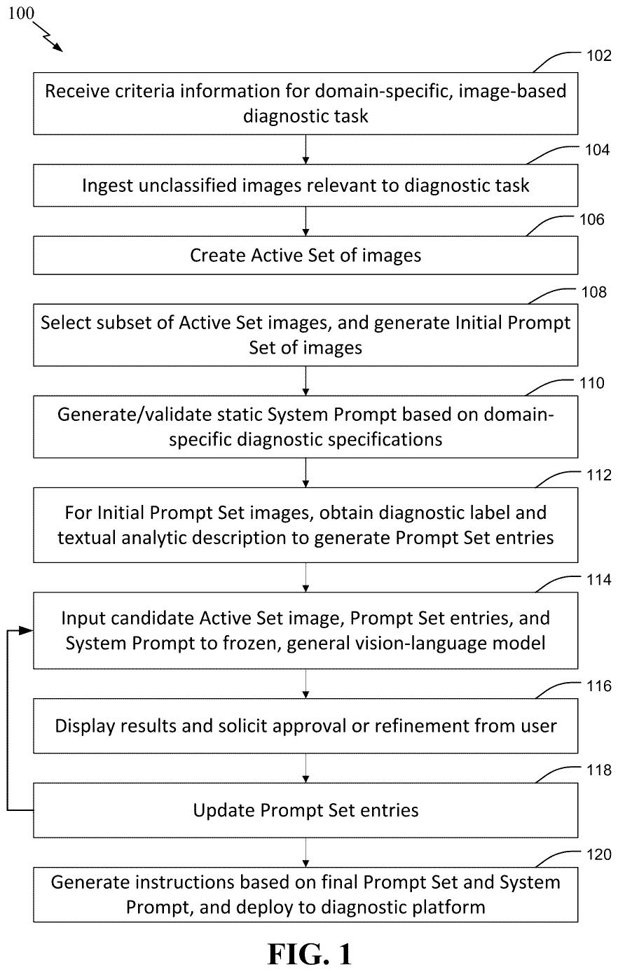

FIG. 1 illustrates an example process 100 for utilizing an active prompt tuning algorithm in the context of a domain-specific, image-based diagnostic task, to develop a framework for consistent and accurate interactions with a frozen vision-language model. Process 100 may be implemented by a software platform of one or more applications or routines executing on one or more computing devices, such as a cloud-based diagnostic platform, a local workstation, or a distributed inference engine. In some embodiments, process 100 may be implemented as a set of software modules or data analysis services operating within an image analysis suite, such as in a medical, veterinary, or other diagnostic lab.

Process 100 may include block 102, which may entail receiving information that defines the scope, criteria, goal, and/or parameters of a domain-specific image diagnostic task. In some embodiments, this information may include a set of possible diagnostic labels, information on suitable or usable imaging modalities or modality specifications (e.g., resolution, noise ratios, image orientation or perspective, magnification, coloring method, anatomical region, etc.), and task-specific constraints or goals (e.g., diagnostic accuracy thresholds, interpretability requirements).

In some embodiments, block 102 may entail retrieving the information defining the task from one or more external sources. For example, input images (see block 104) may comprise metadata that describes their modality, content, methods of acquisition, domain-specific metadata from a trusted, expert knowledge base or receiving user input via a graphical user interface (GUI). DICOM images are a well known example of medical images that may contain metadata that can define some or all of a diagnostic task (or from which the criteria for such task could be derive din whole or in part). Other examples may include more common image formats such as JPEG/PNG with EXIF data or customer header information (such as might be used in dermatology, telemedicine applications, etc.). Alternatively (or in addition), such information may be obtained in whole or in part from a user selection, user input, or from a secondary search tool for obtaining domain information (e.g., another LLM with access to diagnostic protocol information, a library of task criteria for various classification or diagnostic applications, etc.).

The received information may be stored in a task configuration object that is referenced throughout the remainder of process 100. For example, the received information may be formatted into a diagnostic definition object, which may include one or more fields such as a diagnostic label schema, imaging modality descriptors (e.g., type of modality, image acquisition settings, etc.), anatomical region (or other object of interest) identifier, composition of the image (e.g., pose, orientation, plane, exposure, thickness, resolution, color channels, geolocation, etc.), methods of obtaining the image (e.g., contrast-enhanced MRI of given settings vs. non-contrast), decision-specific criteria (e.g., output formats, requirements of input data sufficiency, number of images desired or required, content of images, permissible types of images, etc.), and/or application and system-specific constraints (e.g., minimum prompt set size, explanation length, number and types of vision-language models to be used, output formats, etc.). The task definition object may be stored in memory and referenced by downstream threads, applications, and/or processes to configure the system prompt, validate user input, filter candidate images, and structure model inference requests throughout process 100.

Process 100 may further include block 104, which may entail receiving or ingesting a set of digital images that are relevant to the diagnostic task defined in block 102. (E.g., images of a type that would customarily be used to perform a diagnosis or classification in the given domain and/or would serve as inputs to a novel diagnostic approach as described herein). In some embodiments, the images may be received from a user upload, a mobile device, a telemedicine application, a connected imaging device, a laboratory information management system (LIMS), a picture archiving and communication system (PACS), an electronic medical record, a DICOM server, an Internet-enabled image or web search, video files, or a remote data repository.

In some embodiments, block 104 may further entail parsing or extracting metadata from the received images themselves, or from related context or other information sources associated with the images. For example, DICOM images may include metadata fields such as Modality, Body Part Examined, Study Description, and Image Orientation, among others. As another example, metadata for images may be drawn from other metadata fields and/or from websites or repositories from which the images were obtained. These metadata fields may be used to validate the relevance of the image to the diagnostic task, to filter or group images by modality or subject (e.g., anatomical region), or to populate fields in the task definition object described in block 102. Similarly, microscopy and slide image formats (e.g., SVS, NDPI) may include metadata describing magnification, staining method, or acquisition parameters, which may be used to determine whether the image is suitable for inclusion in the diagnostic task. Other image formats are also known to contain metadata useful for defining at least a portion of a diagnostic or classification task, such as sonar imaging, satellite imaging, etc.

In some embodiments, block 104 may also include preprocessing operations on the received images. For example, the system may crop or re-size images to ensure compatible image dimensions, adjust color gamut and other visual settings for consistency, segment or threshold pixels or voxels according to any of their properties (e.g., brightness, color channels, similar groupings, etc.), apply noise reduction or contrast enhancement, or extract image tiles or patches from larger images according to specified positions or content criteria (e.g., excluding blank areas, defining patches by information density, etc.). These preprocessing steps may be performed automatically or based on parameters defined in the task definition object. In some cases, the system may also perform quality control checks to exclude images that are corrupted, incomplete, or otherwise unsuitable for diagnostic analysis.

The received and optionally preprocessed images may be stored in a structured format, such as a database, object store, or in-memory data structure, and may be associated with metadata tags or identifiers that link them to the diagnostic task or a set of possible compatible diagnostic tasks.

At block 106, process 100 may determine or create an “Active Set” of images from the received images. The images chosen for the Active Set may comprise images that are unclassified (e.g., according to the diagnostic schema of the task definition object) and have not yet been used to construct prompt examples. The Active Set may be stored in a queue, buffer, or other data structure for iterative processing in subsequent blocks of process 100, or may remain in the same memory as the other received images but with a flag or marker indicating their status.

In some embodiments, the system may select images for inclusion in the Active Set using random sampling or other statistical sampling techniques. For example, the system may apply stratified sampling to ensure that the Active Set includes images from each imaging modality, anatomical region, or diagnostic label category defined in the task definition object (see block 102).

Alternatively, the system may use clustering or diversity-based sampling to promote overall variation (or weighted variation according to diagnostic relevance) of the Active Set across visual or metadata-derived features. For example, in some embodiments, the system may analyze the received images to identify characteristic distributions or patterns that may inform Active Set selection. For example, the system may evaluate image-level attributes such as resolution, signal-to-noise ratio, color channel composition, frame rate (for video-based imaging), or acquisition modality. The system may then select images that span the range of these attributes to ensure that the resulting prompt set will expose the vision-language model to a broad and informative range of diagnostic contexts.

In some embodiments, the system may prioritize images with high information density or diagnostic relevance (for example, the system may use pixel value variation and distribution, clustering algorithms, or pretrained feature extractors to identify images that contain more visual features of relevance than others (e.g., less whitespace, more color density indicative of a given stain, more contrast or localized variation, etc.). These images may be prioritized for inclusion in the Active Set to improve the quality and generalizability of the prompt set, and may optionally be balanced with images having comparatively average or low information density and diagnostic relevance.

In some embodiments, a user may be prompted to participate or aid in the selection of the Active Set. For example, a system operating with process 100 may present a gallery or list of candidate images and allow the user to select images for inclusion based on visual inspection, metadata filters, or textual descriptions. In some cases, involvement of a human-in-the-loop for selection tasks like this can help emphasize features of interest and reduce bias or irrelevancy. The user may also provide free-text input describing the types of images that should be included (e.g., “include examples with low contrast,” “select images from the left hemisphere,” or “prioritize pediatric cases”), and the system may use natural language processing to interpret and apply these criteria.

In some embodiments, the system may combine multiple selection strategies. For example, the system may first apply statistical sampling to ensure coverage across modalities and anatomical regions, then refine the selection using information density metrics or user-defined constraints. The resulting Active Set may then be structured or ordered (e.g., a queue, list, or indexed collection) in a strategic way that is most likely to generate the most probative prompt set entries and fastest review time. The content and ordering of the Active Set may also be dynamically updated as images are processed, reviewed, or incorporated into (or rejected from) the prompt set in subsequent blocks of process 100. E.g., where images of a certain type do not tend to wind up as useful prompt set entries (e.g., a user regularly rejects them, or regularly provides the same description for all of them, indicating low independent probative value) or improve performance, the content and ordering of the Active Set can be adjusted to deemphasize that type of image.

At block 108, process 100 may then select a subset of the Active Set of images to serve as an Initial Prompt Set. The Initial Prompt Set of images can be used to serve as an unstructured component of a refined or tuned instruction set to a vision-language model, to serve as a verified set of examples that have been confirmed by a desired human-in-the-loop The images of the Initial Prompt Set may be presented to a human user or other expert resource to be given one or more structured diagnostic or classification labels plus an unstructured, visual-semantic textual description of the content and aspects of the image that resulted in the labeling. For example, where the diagnostic task is to review drone images of a concrete load-bearing support beam for deterioration, a human expert may review the images and append one or more structured labels to the image, which may include: global labels of the entire image, like “sound” or “unsound;” labels indicative of several diagnoses or sub-diagnoses of the image, like “exposed rebar from spalling,” “cracking from shear stress,” “overload sagging,” “water migration,”; and/or localized labeling of specific portions of the images that demonstrate the diagnoses or sub-diagnoses. The human expert may also provide unstructured audio or textual description of affirmatively why the diagnoses were made, including domain-specific explanations of environmental, contextual, and visual factors and features that support or require such diagnoses (e.g., “this support beam is in a below-grade parking garage in a region prone to flooding; the photo perspective shows it is a horizontal beam, with discoloration on the underside; the spalling, porosity, and presence of efflorescence indicates likely water migration; thus, the discoloration can be ascribed to rust and deterioration of internal reinforcement.”). In other embodiments, negative unstructured information may also be provided to explain why an image did not receive a given diagnosis or labeling—such information may be solicited from the human-expert via a separate input feature of a user-interface; optionally, via textual suggestions or instructions coincident with instructing the human-expert to provide the affirmative unstructured information; or via a separate request to the user such as in instances in which a vision-language model running transparently in the background might have labeled an image with a label the human-expert did not apply.

The images of the Initial Prompt Set, combined with the structured information (e.g., labeling and related approaches) and unstructured information, are then transformed into Prompt Set entries to be utilized in an improved review/diagnosis procedure as described below.

The number of images of the Initial Prompt Set may be determined by a user, by the human-expert reviewer, or as a minimum threshold number that is optionally increased or supplemented by the user(s) as deemed necessary, valuable, or appropriate by the human-expert reviewer.

Process 100 may include block 110, which may entail generating or validating a System Prompt that provides context and general diagnostic instructions to the vision-language model. The System Prompt may serve as a static or semi-static textual component that defines the diagnostic context, expected output format, and interpretive role of the model during inference. In some embodiments, the System Prompt may be constructed automatically by the system based on the task definition object (see block 102), retrieved from a library of predefined prompt templates, authored or edited by a user, and or developed or supplemented by a separate unfrozen language model (e.g., using retrieval augmented generation based on an approved resource set, such as a library of domain-specific specifications, guides, textbooks, scientific articles, standards, protocols, etc.).

In some embodiments, the System Prompt may include one or more of the following: (i) a role-based instruction (e.g., “Act as a neuropathologist reviewing cresyl violet-stained cerebellum sections”), (ii) contextual information about the environment, diagnostic objective or goal, overall composition of the image, intended subject of the image (e.g., a component or specific feature of a larger object or scene), how samples in the images were prepared, pose of the object/subject of the image, etc.; (iii) image acquisition or setting information, such as imaging modality, camera settings, zoom, frame rate, video duration, stain color, slice thickness or voxel size, resolution, location, angle/perspective, anatomical region, (e.g., “These are 10× magnification images of mouse cerebellum stained with cresyl violet”), and (iv) output formatting constraints (e.g., “Respond with a diagnostic label and a one-sentence explanation of the visual features supporting the diagnosis, in JSON format”). The System Prompt may also include instructions for how the model should interpret the Prompt Set examples, how to handle uncertainty, or how to prioritize certain visual features.

In some embodiments, the System Prompt may be generated automatically or dynamically by parsing metadata from the task definition object and formatting it into a structured prompt template. A structured lookup library may be utilized to associate diagnostic task definition keywords from the task definition object, using natural language processing (with aid of definitions and synonym information), with role instructions. Or, a separate LLM could generate or obtain such information based on the task definition object. For example, if the task definition object specifies a diagnostic task involving 20× magnification images of skin lesions, the system may generate a System Prompt that includes a role instruction such as “Act as a dermatopathologist” and a context statement such as “These are dermoscopic images of pigmented skin lesions captured at 20× magnification.” The system may also include formatting instructions such as “Return the diagnosis as one of: melanoma, nevus, seborrheic keratosis.”

In some embodiments, the System Prompt may be validated or refined by a user. For example, the system may present the generated prompt to the user and/or human-expert (if different) for review and allow the user to edit or approve the prompt before it is used a diagnosis task.

In some embodiments, the System Prompt may be stored in association with the task definition object and reused across multiple diagnostic task cycles (e.g., multiple pathology reviews). The System Prompt may remain static (once validated) throughout process 100, or may be updated if the diagnostic task is redefined, if new metadata becomes available, or if the user modifies the diagnostic criteria. In some cases, the System Prompt may be versioned (such as done with auditable SOPs or design revisions) or checkpointed to support historical review, auditability, or regulatory compliance.

In regard to output requirements, the System Prompt may also include instructions for exactly what information the vision-language model should output as well as instructions for how it should handle unclear, ambiguous or marginal cases (e.g., inconsistent or low confidence levels). For example, the System Prompt may require an organization of model output so that a subsequent software process or module can consistently receive and process the ouputs in an expected way, such as a format for how labels are communicated, whether unstructured explanatory text should be provided and if so how that text should be designated as relating to the image: globally, label-by-label, localized, etc. Similarly, the System Prompt may define how model output should express uncertainty, similar or inconsistent confidence levels, whether additional diagnoses or labels were “close”, and whether to include unstructured textual description of labels that were not used, and/or how to defer to human review. For example, the prompt may include language such as “If the diagnosis is uncertain, return ‘uncertain’ and explain the ambiguity,” or “If the image quality is insufficient, return ‘insufficient quality’ and describe the issue.” These instructions may help ensure that the model's outputs are interpretable, trustworthy, and aligned with clinical or operational expectations, and to notify human users of rationale and confidence in outputs.

Process 100 may further include block 112, which may entail generating Prompt Set entries for each image of the Initial Prompt Set by obtaining both structured diagnostic labeling and unstructured, visual-semantic textual description from one or more human-experts or other qualified reviewer(s). These Prompt Set entries may serve as foundational examples for guiding behavior of a frozen vision-language model as one part of an overall diagnostic process for new, unlabeled images, and may be used in combination with the System Prompt (see block 110) to form a more robust instruction input and consistent output format to coordinate with previous and subsequent modules, applications, threads, etc. of process 100.

In some embodiments, process 100 may obtain the structured and unstructured information from the user through a dedicated user interface specifically configured to serve as a training or tuning phase. This interface may be configured to solicit input from a domain expert-such as a pathologist, structural engineer, radiologist, security analyst, or other qualified reviewer-who is intentionally participating in the creation and refinement of the Prompt Set. The interface may include features for image display, label selection, annotation tools, and free-text entry fields for unstructured explanations. In other embodiments, the process may operate in a transparent or semi-transparent manner, wherein the expert's routine diagnostic or review activities (e.g., labeling, annotating, cropping, or commenting on images) are monitored by a background process or agent that identifies candidate images and associated expert input for potential inclusion in the Prompt Set. For example, while a pathologist is reviewing a set of histological slides and entering diagnoses or annotations as part of a standard case review, the system may detect that certain images and associated inputs meet the criteria for inclusion in the Active Set and may automatically extract the structured and unstructured information for later confirmation and use in the Prompt Set.

In some embodiments, process 100 may deploy an integrated workflow interface that requests a user to identify or confirm which prior diagnostic studies or image reviews (stored from previous work) should be used to generate the Initial Prompt Set. In such cases, the system may automatically extract/load the structured labels and unstructured annotations from those prior studies, and either (i) present them to the user for confirmation and refinement, or (ii) use them directly to initialize the Prompt Set and proceed to iterative prompt expansion. In further embodiments, the system may perform a hybrid approach in which a baseline Prompt Set is generated automatically from historical data, and the human expert subsequently participates in active tuning steps to refine, validate, or expand the Prompt Set based on real-time feedback and model performance.

In some embodiments, the structured diagnostic labeling may include information as described above, including one or more of: (i) a global label for the image as a whole (e.g., “normal,” “abnormal,” “unsound,” etc.); (ii) one or more specific diagnostic labels or sub-diagnoses (e.g., “efflorescence,” “shear cracking,” “tumor infiltration,” “necrosis,” etc.); and/or (iii) localized labels or annotations that identify specific regions or features of the image associated with the diagnosis. The structured labels may be selected from a predefined set of labels defined in the task definition object (see block 102), or may be entered manually by the user and subsequently validated or normalized by the system.

In addition to the structured labeling, the system may prompt the user to provide an unstructured textual explanation of the image, describing the visual, contextual, and/or domain-specific features that support the assigned diagnostic label(s). In some embodiments, the system may present the user with the same contextual information that is included in the System Prompt (e.g., “These are 10× magnification images of mouse cerebellum stained with cresyl violet”) to ensure that the user's explanation is appropriately focused and does not reiterate baseline information already provided to the model. This may help the user concentrate on describing the diagnostic reasoning, visual cues, and domain-specific interpretations that are not otherwise encoded in the System Prompt.

In some embodiments, the system may provide guidance, templates, or examples to assist the user in composing informative and interpretable explanations. For example, the system may prompt the user with instructions such as: “Describe the visual features that support the diagnosis,” “Explain why this image does not show signs of pathology,” or “Indicate any contextual factors (e.g., location, environment, acquisition method) that influenced your diagnosis.” The system may also support the entry of negative or contrastive explanations, such as why a particular diagnosis was not applied, or why a commonly confused condition was ruled out.

In some embodiments, process 100 may validate the user-provided inputs for completeness, formatting, or consistency with the task definition object. For example, block 112 may provide the reviewer with information from the System Prompt to give context, check that the selected diagnostic label is valid for the current task, that the explanation meets a minimum length or content threshold, or that the explanation includes references to expected anatomical or structural features. Process 100 may also support optional review or approval workflows, in which a second user or domain expert verifies the prompt entries before they are finalized.

Each Prompt Set entry may be stored as a structured data object comprising the image, the structured diagnostic label(s), and the unstructured textual explanation, along with any additional image-specific metadata that should be assessed by the model. In some embodiments, the Prompt Set entries may be stored in a format compatible with the input requirements of the vision-language model, such as a JSON array of image-caption pairs, a tabular structure with fields for image metadata, ID, label, and explanation, etc.

In some embodiments, block 112 may include associating each Prompt Set entry with contextual metadata from the image or task definition object, such as imaging modality, acquisition parameters, or anatomical region, which will be provided to the model during diagnostic tasks. The resulting Prompt Set may be used in subsequent blocks of process 100 to guide model inference and iterative prompt expansion.

Process 100 may further include block 114, which may entail selecting a candidate image from the Active Set and submitting it, along with the Prompt Set and the System Prompt, to a frozen vision-language model for diagnostic inference. The frozen vision-language model may be a pretrained, general-purpose model, eliminating the need for a large amount of training data which would typically be needed to fine tune or retrain such a model to become a specific-purpose model. The frozen vision-language model may thus be a multimodal transformer network (e.g., capable of inputting image/video and text information), or other large-scale model architecture capable of processing both visual and textual inputs, and may be accessed via a local inference engine, a cloud-based API, or a distributed/federated computational infrastructure.

In some embodiments, the candidate image may be selected from the Active Set using a predetermined sampling strategy, such as random sampling, stratified sampling, or prioritization based on metadata attributes (e.g., modality, resolution, anatomical region, or acquisition method) as described above with respect to ordering of the Active Set. The system may also apply heuristics or learned policies to prioritize images that are expected to yield high diagnostic value or that represent underrepresented or unique categories in the current Prompt Set.

Once selected, the candidate image may be combined with the current Prompt Set and the System Prompt by a software module within the overall pipeline, to form a composite input to the vision-language model. The Prompt Set component of the composite input may be all or multiple entries, as described in block 112. The System Prompt may be as described in block 110. Together, these components may be formatted into a structured input sequence by a module of process 100 to conform to the model's expected input schema (e.g., a JSON object, a tokenized prompt block, or a multimodal input stream). The module of block 114 may also (e.g., transparently to the user) experiment with random re-ordering/re-sequencing/adjustment of the Prompt Set component to attempt multiple permutations and assess whether doing so meaningfully affects model output.

Thus, in some embodiments, the system may include a prompt assembly module or preparation engine that dynamically constructs model input by serializing permutations of the Prompt Set entries and appending the System Prompt and candidate image. Such modules may also apply input length constraints, truncation policies, or prompt compression techniques to ensure that the input remains within the model's token or memory limits.

Upon receiving the composite input, the frozen vision-language model may generate one or more outputs, including: (i) a predicted diagnostic label for the candidate image, and (ii) a generated unstructured explanation describing the visual features or contextual factors that support the predicted diagnosis. In some embodiments, the model may also output additional metadata, such as confidence scores, uncertainty flags, or alternative diagnoses. The model's outputs may be formatted according to the instructions specified in the System Prompt, and may be returned for further processing, display, or review.

Alternatively, multiple frozen vision-language models may be provided the composite input (or customized composite inputs tailored to the capabilities of the models) to seek an optimized workflow. For example, where a lightweight solution is desired, models of various parameterizations/weight sizes might be tested. Or, where accuracy and interpretability are desired, a group of several models may be queried as an ensemble.

In some embodiments, block 114 may log the model's outputs along with the input components for use in downstream activities of process 100, and/or for auditability, reproducibility, or future analysis.

Process 100 may include block 116, which may entail processing output of the vision-language model(s) and generating a presentation thereof for a human user to review, and soliciting feedback in the form of approval, rejection, or refinement. The outputs presented to the user may include the candidate image, the predicted diagnostic label(s), and the unstructured textual explanation generated by the model in response to the Prompt Set and System Prompt (see block 114). In some embodiments, the system may also display additional metadata or model-generated information, such as confidence scores, uncertainty indicators, information about the characteristics and training of the model(s), or alternative diagnoses.

In some embodiments, the system may present the candidate image and model outputs within a dedicated user interface configured for expert review. The interface may include tools for visual inspection (e.g., zoom, pan, contrast adjustment), structured feedback options (e.g., radio buttons or dropdowns for “approve,” “reject,” or “approve with edits”), and free-text input fields for refining or replacing the model-generated explanation. The interface may also include contextual information from the System Prompt (e.g., imaging modality, anatomical region, acquisition parameters) to assist the user in evaluating the model's outputs in the appropriate diagnostic context.

In some embodiments, the system may allow the user to add/remove/edit the model-generated diagnostic label or explanation directly, or to annotate the image with additional information (e.g., highlighting regions of interest, adding comments, or attaching references). The system may also support structured refinement workflows, in which the user is prompted to confirm or revise specific elements of the model's output (e.g., “Is the diagnosis correct?”, “Does the explanation reference the correct anatomical structure?”, “Would you like to add a clarification?”).

In some embodiments, the system may support multiple modes of feedback collection. For example, in a training or tuning phase, the user may be explicitly prompted to review and refine each model output in detail. In a semi-transparent or background mode, the system may monitor the user's routine diagnostic activities (e.g., confirming or overriding model suggestions, entering new labels or explanations) and infer feedback based on those actions. In either case, the system may log the user's feedback and associate it with the candidate image and model output for use in subsequent blocks of process 100.

In some embodiments, the system may also support collaborative or multi-reviewer workflows, in which multiple users (e.g., multiple pathologists, reviewers from different specialties, etc.) provide feedback on the same model output. The system may aggregate or reconcile the feedback using predefined rules or consensus mechanisms, and may present the final decision to a user for confirmation.

The feedback collected in block 116 may be used to determine whether the candidate image and associated information should be incorporated into the Prompt Set, and if so, in what form.

Thus, at block 118, process 100 may involve determining whether to update the current Prompt Set to incorporate the candidate image and its associated diagnostic label and unstructured description, based on the feedback received from the human-expert or user in block 116.

In some embodiments, process may determine whether the feedback provided in block 116 constitutes an approval, a rejection, or an approval with refinement. If the feedback indicates approval of both the diagnostic label and the explanation, the system may directly incorporate the candidate image and associated information into the Prompt Set. If the feedback includes refinements or corrections—such as a revised label, an edited explanation, or additional annotations—the system may incorporate the corrected information into the Prompt Set in place of the original model output. If the feedback indicates rejection, the system may discard the candidate image from the current inference cycle or flag it for further review, exclusion, or deferred processing.

In alternative embodiments, where accuracy and human verification is exceptionally important, process 100 may utilize a double/multi-confirmation algorithm. For example, process 100 may store a user's ‘approval’ of a candidate image, but then still randomly re-present that same image with the same label and the same (or reworded) unstructured description so that the human expert approves the image and explanation twice before it can become part of the Prompt Set. In some cases, process 100 may perform an image modification or augmentation on candidate images from the Active Set, and admit the candidate to the Prompt Set only if all pairs/members of a modified/augmented image set receive the same diagnosis and a similar unstructured description. For example, images may be rotated, mirrored, cropped, brightened/darkened, or otherwise modified in ways that are known not to affect diagnosis/categorization based on the domain of the diagnostic task (e.g., changes in color may not be suitable for infrared images, cropping may be suitable for certain types of medical images, brightness changes may be suitable for survey drone videos, etc.). In other alternatives, two separate human experts may need to approve a candidate/information pair before it can become a Prompt Set entry; or a second human expert may need to approve a revision to the unstructured description.

In some embodiments, process 100 may apply additional logic or constraints when updating the Prompt Set. For example, in a similar fashion to how images were selected for the Active Set, a monitoring algorithm may enforce diversity constraints to ensure that the Prompt Set does not become overly biased toward a particular diagnostic category, modality, or visual pattern. The system may also apply quality control checks to ensure that the unstructured explanation meets minimum standards for clarity, completeness, or domain relevance. In some cases, the system may prompt a second reviewer to confirm the inclusion of a new Prompt Set entry, or may flag entries for periodic review or revalidation.

Once an image and associated information are confirmed, block 118 may store the updated Prompt Set entry as a structured data object, including fields for the image, the final diagnostic label(s), the final unstructured explanation, and any associated metadata (e.g., reviewer ID, timestamp, confidence score, or rationale for inclusion).

As new Prompt Set entries are added, block 118 may optionally track the content and changing composition of the Prompt Set over time, including the number of entries, the distribution of diagnostic labels, and the diversity of visual features represented. This information may be used to determine whether additional Prompt Set entries are needed, whether the Prompt Set is sufficiently representative for the diagnostic task, and recording heuristics such as how long it takes a human expert to generate a suitable Prompt Set and how large a Prompt Set should be (for use in future refinement tasks).

As each Prompt Set entry is added, process 100 may determine whether to continue generation of more Prompt Set entries (e.g., performing another iteration of block 114-118) or ceasing further modification of the Prompt Set entry and moving to deployment.

A variety of algorithms for making this determination are contemplated. First, when the predicted diagnosis and explanations for candidates of the Active Set have been accurate/approved a given number of instances in a row, or exhibit a high accuracy (e.g., overall high rate of approval), further iterations of steps 114-118 may cease. Depending upon the manner in which process 100 is coordinating and soliciting human feedback, it may implement various different algorithms for determining when or how to cease soliciting feedback. Thus, in operation, embodiments of process 100 control how the final software tool is generated, how it behaves, and how it is deployed, in part by controlling how human-in-the-loop involvement takes place: process 100 controls which images are presented to a human expert(s), which structured and unstructured feedback is solicited from the expert(s), the size of the Prompt Set, criteria and rigor for adding entries to the Prompt Set, the composition of the Prompt Set relative to the Active Set, etc.

For example, where the diagnostic task involves multiple possible diagnostic labels (such as in the example discussed above of structural engineering image review, or in the case of skin lesion diagnosis), process 100 may monitor the distribution of labels represented in the Prompt Set entries and determine that iteration should cease once each label is represented by a minimum number of examples (which may be an equal number across labels or may correspond to reported data on occurrence). Similarly, where the diagnostic task involves multiple imaging modalities, anatomical regions, or acquisition conditions, process 100 may ensure that the Prompt Set includes representative examples from each relevant category. Thus, in cases where process 100 determines that the Prompt Set does not contain an acceptable number of entries representing a given label or condition, when process 100 next repeats block 114 an image corresponding to the under-represented condition or an image likely to have the given label can be prioritized for next review.

In other embodiments, process 100 may apply performance-based criteria to determine whether further iteration is likely to yield meaningful improvements. For example, process 100 may track the effect of each new Prompt Set entry on the model's output consistency, accuracy, or interpretability. If the addition of new entries fails to produce measurable gains in model performance—such as improved agreement with expert review, reduced uncertainty, or increased diagnostic specificity—process 100 may determine that the Prompt Set has reached a point of diminishing returns. In some implementations, this determination may be based on a moving average of model performance metrics, a convergence threshold, or a statistical test of marginal utility. Relatedly, where the unstructured visual-semantic information being generated by the vision-language model exhibits repeating word choices, descriptors, or discussion patterns in a domain in which such repetition is not helpful or could engender distrust, process 100 may require further review to take place on images likely to be distinct from those already reviewed and/or may remove similar images from the Prompt Set. On repeating block 114, process 100 may actively solicit different descriptions from the human expert, such as querying the expert regarding domain-specific diagnostic criteria (e.g., derived from the System Prompt, task definition object, or trusted expert resources) and enforcing discussion of both the presence and absence of visual features pertinent to all or multiple of those criteria.

In further embodiments, process 100 may emphasize human validation as a criterion for finalizing the Prompt Set. For example, process 100 may require that a minimum number of Prompt Set entries be reviewed and approved by multiple human experts, or that a subset of entries be independently confirmed by different reviewers, before closing the Prompt Set. In some cases, process 100 may require that each expert review both overlapping and non-overlapping subsets of the Prompt Set, to ensure both consistency and breadth of validation. Alternatively, process 100 may implement a double-confirmation protocol, in which independent criteria (e.g., performance criteria, representative distribution, number of entries) are used to initially define a Prompt Set, but then each image/information pairing is subsequently re-confirmed be being presented tor represented to the same or different reviewers (in a different order, in different subsets/combinations, at different times, using modification/augmentation, etc.) the Prompt Set is only finalized if these ‘second’ reviews yield consistent approval. In some embodiments, process 100 may also incorporate adversarial or contrastive review, in which reviewers are asked to identify potential weaknesses, ambiguities, or alternative interpretations of Prompt Set entries (without knowing whether they were generated by the vision-language model entirely, by another human expert entirely, or by a human expert's modification of the vision-language model's prediction), and iteration continues until all disagreements and inconsistencies are resolved or no longer arise.

In still other embodiments, process 100 may combine multiple criteria-such as label distribution, performance convergence, and expert consensus-into a composite stopping condition. For example, process 100 may require that a minimum number of Prompt Set entries be approved by at least two reviewers, that the Prompt Set span all diagnostic labels and modalities defined in the task definition object, and that the model's outputs stabilize across a validation set of candidate images. Once these conditions are satisfied, process 100 may proceed to block 120.

Other variations for automated development, expansion, and finalization of the Prompt Set are also contemplated, including combinations and variations of the foregoing as well as domain-specific criteria. For example, where the diagnostic task involves identification of disease or infection, additional features that are presented over time (e.g., growth patterns, growth rate, etc.) or in additional dimensions (e.g., vibration frequency of an object in an image) may also be used as criteria for expanding or closing the Prompt Set.

Moreover, a domain-specific monitoring of runtime input data features may also prompt process 100 to determine whether to adjust the Prompt Set. For example, after deployment, an input pre-processing module may compare statistics of input data to determine if the input set is aligned with only a subset of the Prompt Set entries (e.g., based on image modality, anatomy-type, etc.) and dynamically prune and/or supplement the Prompt Set accordingly. Additionally, as input images start to exhibit under-represented or novel features relative to the Prompt Set entries, a software pre-processing module may re-open some or all of the steps of process 100 (including human-expert-in-the-loop validation) for such new images.

Relatedly, it is contemplated that process 100 may iteratively refine the System Prompt (e.g., by rewording or rephrasing) based on edits by a human reviewer, feedback from a human reviewer solicited during iterative development of the Prompt Set, or automatically in a background process, to optimize the System Prompt relative to the evolving Prompt Set. Thus, as each entry is added to the Prompt Set, process 100 may provide several instruction sets to the frozen vision-language model using the expanded Prompt Set to ascertain whether the results differ in a meaningful way. Where appreciable differences are identified, process 100 may transparently present the different predicted labels/unstructured text to the human user to determine relative accuracy, or may present the different outputs simultaneously for the human user to select the preferred or correct output.

And, in addition to or instead of refining the System Prompt, process 100 may also utilize more than one frozen vision language model when processing candidate images of the Active Set, and track cumulative accuracy, rate of edits by the human experts, etc. to determine which model provides best output from a standpoint of performance, cost, resource demand, etc.

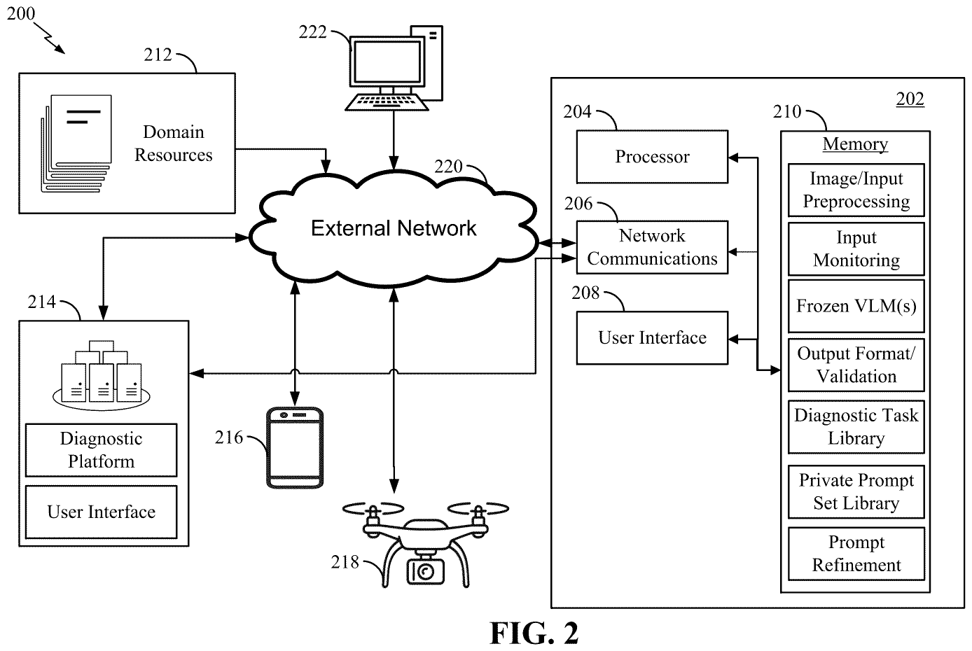

At block 120, process 100 may integrate the Prompt Set and System Prompt into a software application environment or system for performance or supplementation of an image-based diagnostic task (such as depicted in FIG. 2). Thus, block 120 may involve an operational deployment and custom integration phase, and development or adaptation of software processes or modules to manage data preprocessing, monitoring, and input as well as to manage formatting, transmission, and review of output.

For example, upstream modules may normalize image metadata, convert proprietary formats, or extract relevant regions of interest to match the input expectations of the VLM. Downstream modules may translate the model's outputs-such as classification labels, explanatory captions, or similarity scores-into structured reports, annotations, or alerts that can be consumed by the host platform. These modules may be implemented as part of process 100 or may be provided by the host platform itself.

The host platform may include, but is not limited to, commercial diagnostic review suites such as Philips IntelliSite Pathology, Leica Aperio eSlide Manager, or Sectra PACS; web-based portals for remote image review; custom-built software applications; or image search engines that support case retrieval and triage. In each case, the Prompt Set and System Prompt serve as a modular inference layer that can be embedded within or alongside the host platform to enable consistent, explainable, and context-aware outputs from the VLM.

### Example Hardware Configuration

Referring now to FIG. 2, a block diagram is shown illustrating a system architecture 200 for implementing and performing the methods described herein (including, e.g., with respect to FIG. 1). The system 200 includes a computing device 202, which may perform some or all of the steps of the methods described herein. Device 202 includes a processor 204, memory 210, a network communications card (e.g., network card, cellular transceiver, ethernet module, etc.) 206, and a user interface 208 (which may include a display/monitor, keyboard, etc., and ports for connection to the same). Processor 204 may be a general-purpose processor, or an application-specific chip, but will in at least some embodiments be sufficient in resources and computational power to operate a companion graphics processing unit (GPU). In many embodiments, the size and nature of the image sets to be processed may warrant a custom processor be utilized, such as those used to operate DICOM systems or the like. The GPU may be a general GPU or may be customized in resources (or even tailored in its transistor level layout) to run a frozen or controlled vision-language model. Because one aspect achievable by the present systems and methods is elimination or mitigation of a need to retrain or fine-tune a vision-language model (e.g., adjust weights, run training operations, etc.), a special-purpose GPU can be utilized to maximize efficiency, performance, and resource utilization of the frequent calls to the VLM described herein.

In some embodiments, device 202 may be a standalone server, cloud resource, or computing resource, while in other embodiments it may be integrated into a diagnostic platform 214, such as a digital pathology viewer, radiology PACS, DICOM viewer, or laboratory information management system (LIMS). In either case, device 202 may operate locally or communicate with external systems via network 220.

Memory 210 may store data sets (including previously reviewed and/or to-be-reviewed images), but may also store several software applications, functions, routines, and/or programs that execute various functions related to image-based diagnoses using a frozen vision-language model (VLM). These include an image/input preprocessing module, an input monitoring module, one or more frozen VLMs, an output formatting and validation module, a diagnostic task library, a private prompt set library, and a prompt refinement module.

The image/input preprocessing module (e.g., function, application, script, software, etc.) may be configured to receive input images and associated metadata, determine the diagnostic task or request, and assess whether the input images are suitable for that task. This module may normalize the input images, perform quality control, and select an appropriate Prompt Set and System Prompt from the private prompt set library. In some embodiments, the preprocessing module may be dedicated to a specific diagnostic task, while in other embodiments it may dynamically interpret task requests (e.g., using natural language processing and/or a separate LLM call, as described above) and determine whether a matching task definition, Prompt Set, and System Prompt already exist or whether new ones should be created.

The input monitoring module may optionally operate in parallel with main diagnostic tasks performed by device 202, to evaluate whether the input images, while nominally matching a known diagnostic task, are trending toward differences in content from those used to generate the Prompt Set or otherwise include examples that deviate from the Prompt Set. Thus, a special-purpose co-processor or GPU may operate to perform feature extraction and comparison determinations between images in the private prompt set library and incoming input images and/or trends in labelling output of the VLM relative to input images. If a change in content of input images is detected or a change in diagnostic output is detected, the system may initiate a refinement process or branch the Prompt Set to accommodate the new input distribution. This refinement may be performed by the prompt refinement module, which may operate as described above with respect to FIG. 1.

The frozen VLM(s) may be general-purpose, pre-trained VLM(s) or may be fine-tuned or specially trained for diagnostic analyses of images. The VLM(s), however, may be “frozen” in the sense that their weights and training are controlled or controllable by the operator of device 202, and are not subject to a third party's updating, retraining, modification, etc. By having these VLM(s) be “frozen” or only controllably updated, greater consistency and confidence can be achieved with fine-tuned instruction sets (e.g., Prompt Sets+System Prompt pairings). Likewise, massive data sets and massive computational resources are not needed to improve accuracy and behavior of the VLM(s)—instead, users can fine tune the human-interpretable aspects of their behavior such as input formatting, prompt content, etc.

Thus, software stored in memory 210 may automatically modify queries and inputs made to the VLM(s) in ways that are not visible to users. For example, if a radiology lab submits an image set for diagnostic analysis to a diagnostic platform operating with device 202, the lab staff's request may simply be for the diagnostic platform to label a new set of radiology images as exhibiting a type of cancer or not. Software running on device 202 may then transform that request into a structured query to a frozen VLM, by defining or selecting a System Prompt and providing a corresponding Prompt Set along with the to receive the preprocessed input along with the new radiology images (and in some cases may even reformat the new radiology images to conform to a particular task definition).

Similarly, the output formatting and validation module ensures that the output conforms to the expectations of the requesting platform, including formatting the output for compatibility and validating that the output includes recognized labels or falls within expectations for diagnostic labels to be used, rate of incidence of diagnoses, consistency in use of labels for images from the same sample/acquisition, as well as review of descriptive text relative to the task definition object to ensure the VLM has not generated inapplicable.

The diagnostic task library and private prompt set library may be stored locally in memory 210. In some embodiments, the prompt set library is encrypted and private, such that Prompt Set examples are never exposed to the client or requesting platform. This configuration is particularly advantageous in privacy-sensitive domains such as healthcare. For example, where images acquired as a part of patient healthcare cannot readily be de-identified (or have not been), they could still be used to form entries in a Private Prompt Set, which is utilized to transform and supplement a diagnostic task for another patient/clinician, without improper disclosure of that patient's information.

Device 202 may communicate with a variety of external systems via network 220. These may include mobile devices 216 configured for user-updatable classification or augmented reality (AR) applications that make rely on device 202 for image diagnosis/classification purposes; autonomous or remotely operated vehicles 218 that may benefit from auditable and validated classification decision-making platforms for images they acquire; even or web-based image search platforms 222 where users seek to control or customize search results based on visual input. In each case, device 202 may operate as an API endpoint, a locally executing module, or a hybrid service depending on deployment constraints.

As noted above, a healthcare organization, electronic medical record system, laboratory, or other organization 214 may operationally interact with device 202 in several ways. For example, device 202 may be part of an internal-network resource of the organization 214, so as to avoid concerns over sharing of private information and/or bandwidth and resource constraints on sending large image files outside of the organization's network. In some implementations, the organization 214 may operate its own image-based diagnostic platform for reviewing medical and laboratory images, which may include its own user interface and software suite. The functionality of device 202 may thus be embedded into an existing image-review suite. In other implementations, device 202 may be an external resource that is operationally connected via an external network 220. In such instances, the organization 214 may have a large image data storage resource, and send desired images remotely to device 202 for analysis.

Furthermore, as device 202 may perform some or all of the steps of FIG. 1 (e.g., for development, modification, or refinement of a Prompt Set and System Prompt for a new or changing diagnostic task), it may also communicate with a trusted source 212 of domain knowledge and resources, such as approved diagnostic protocols and test standards. In some embodiments, a client organization 214 may determine and curate the domain resources to be relied upon, while in other embodiments, device 202 may select such resources automatically.

### Example Implementations and Experiments

The inventors conducted several studies and experiments validating the approaches described herein. One such experiment is described below, to serve as an illustrative example of the improvements achieved by embodiments of the present disclosure in time (both in generating fewer ground truth data examples and in time spent training a large model), resource savings (vs. conventional approaches to training or retraining models like CNNs and fine-tuned VLMS), and human-in-the-loop confirmability and influence on behavior.

Vision Language Models (VLMs) are a category of generative AI with the capability to understand, interpret and analyze both text and images in concert. Thus, VLMs have the ability to leverage knowledge from one modality (images/visual domain) to inform the analysis of the other (textual/semantic domain) and vice versa. Most VLMs incorporate separate encoders for images and text then use contrastive learning (e.g., CLIP) to capture the association between the text, e.g., a phrase/keyword/sentence, and an image. This approach provides learning of cross-modal representations by maximizing the similarity between matched image-text pairs while minimizing the similarity between unmatched pairs.

GPT-4 omni (GPT-4o, for short), is a recent multi-modal model from OpenAI. As discussed below, following combined inputs of text (tokens) for corresponding images in a prompt set, GPT-4o generates output in the form of predicted diagnoses with text-based explanations for the given diagnosis of each image. Other leading models include GPT-4 (V), Claude Opus, Gemini 1.0 Ultra, Gemini 1.5-Pro, as well as vision models trained on specific image-text pairings like BioMedCLIP.

In one study, the inventors utilized GPT-4 (Vision) as the VLM in an active prompt tuning approach for diagnostic categorization of Iba-1 immuno-stained microglia cells in the hippocampus of tissue sections, through mouse brains treated with either a powerful neurotoxin (tri-methyl tin) or saline. Results established an extremely high reduction in human expert time spent annotating slide-images vs manual review as well as an extremely high reduction in number of training data pairs needed before suitable accuracy was achieved.

In a more recent study, the inventors evaluated an Active Prompt Tuning approach on a dataset of low-magnification (10×) images of cerebellum sections of mouse brain stained with cresyl violet, a relatively low signal: noise general marker for all brain cells. This dataset is from 18 mice: images from 9 Lurcher mutant and 9 wild-type (controls) to assess the prediction accuracy of the diagnostic software platform developed around refined/tuned prompting for GPT-4o. Notably, the ground truth examples used in the study represented only a small subsample (2%) of the dataset from all 18 mice. Improvements are described below for accuracy and time required to prepare the ground truth for the classification of two diverse datasets at different magnifications using the APT approach compared to a traditional CNN approach.

In preliminary experiments conducted during a pilot study, we explored different prompting strategies, ranging from zero-shot prompting to varying levels of manual intervention and scope of examples provided via prompting. Empirically (and somewhat unexpectedly, given the advanced power of GPT-4o), we observed some degree of manual correction of the prompts was significantly beneficial toward ensuring that the software produces usable results—in some tests, lack of human expert active correction or provision of prompt example language rendered outputs unusable for these domain-specific tasks. Recent research suggests that the wide variety of types of images used to train these VLMs makes them highly sensitive to input prompts.

Although input prompts that are completely written by a human expert were used for only a small subset of the dataset, manually curating these prompts is a time- and labor-intensive process that demands experts from the problem domain (neuroscience) and linguistics. To address this issue, the inventors utilized a particular “Active Prompt Tuning” approach to not only control refinement of model behavior but also to automate much of the prompt set generation.

In one aspect, active prompt tuning (APT) can be described as an active-learning-based human-in-the-loop approach for selecting the most effective prompt set for a given task. As applied to the inventors' experiments, the software and pipeline that leveraged APT to develop a prompt set, transformed queries using the prompt set, interacted with a VLM, and managed output, will be referred to herein as APT-USF.

In the inventors experiments and prototype platforms, a random subset of images is selected from the dataset according to basic criteria. For example, here we selected 6 random images from each of the 6 (out of 18) random mice set aside for prompting. This subset was further divided into the “active set” and the “initial prompt set”. Under the supervision of a domain expert, a short caption was created for each image in the initial prompt set. These short captions provided the model with clear descriptions of the visual cues in the image, enabling it to classify the images in the test set effectively. In effect, the prompt set becomes a refined instruction to the model to leverage its vast knowledge in a very specific way, within constrained and unconstrained parameters, to the task of diagnostic analysis of particular medical images. The active prompt refinement both ensures the model generates accurate outputs in the expected format, as well as injects human intuition and expert reasoning into model behavior in a customizable and personalizable way.

Each caption used in the study comprised two parts: (1) a ground truth, verified classification of the sample image; and (2) a brief visual-semantic, unstructured, textual explanation highlighting key morphological features that the expert found to support the ground truth classification. These “image-caption pairs” were initially generated by the human domain expert, to form the initial prompt set. Then, the GPT-4o model is prompted to generate captions for images in an active set, using the initial prompt set as a few-shot prompting example. The model input includes both the image-caption pairs from the initial prompt set and one or more images from the active set. By applying in-context learning to the active set of images, the model generates outputs having two items for each image: 1) the predicted classification (e.g., Lurcher vs. wild-type); and 2) a brief (1-3 sentence) visual-semantic, textual explanation for that classification decision.

Next, the correctly classified images from the active set were reviewed by a human expert who verifies and, if necessary, corrects and/or refines the captions. Once verified, the image-caption pairs were added to the initial prompt set. This process repeats for multiple rounds, with each round potentially adding more correct and detailed classified samples from the active set to the initial prompt set, each having embedded therein any additions, corrections, or refinements that incorporate the human expert's insights, reasoning, and domain expertise. The rounds continue until all images in the active set are correctly classified and moved to the initial prompt set. In cases where certain images in the active set are not predicted correctly after several rounds, a threshold (5 rounds) was set to stop the process and prevent it from running indefinitely.

Once the iterative refinement aspect of APT-USF is complete, the initial prompt set becomes the “effective prompt set” that is used as part of the instructions given to the model for diagnoses on the yet unseen test set. This approach significantly reduces manual overhead as only the initial prompt set requires detailed ground truth preparation. For the active set, ground truth preparation is reduced to a verification step, and thus a major improvement in efficiency is achieved.

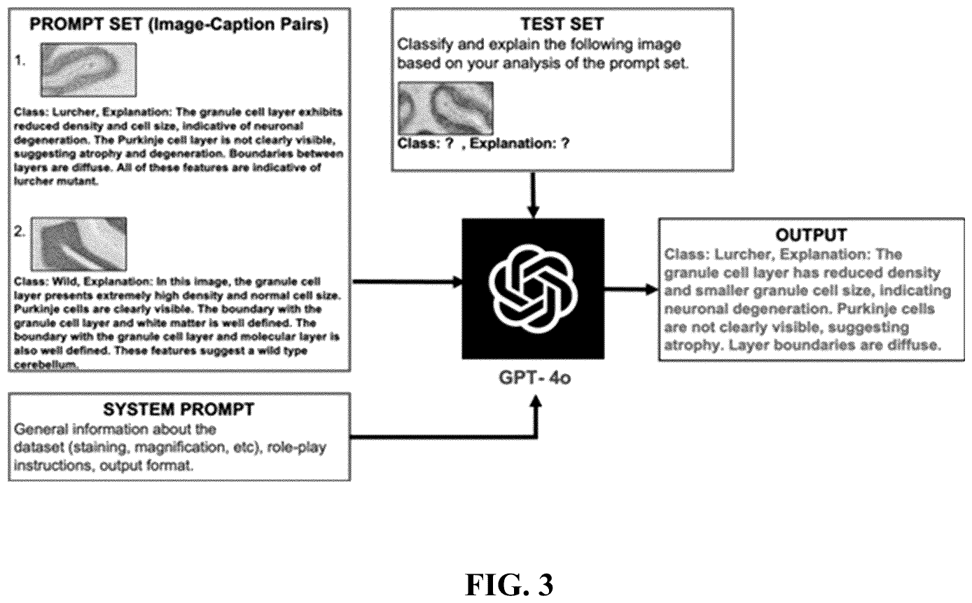

In addition to the prompt set, we developed a “system prompt” to manage, enforce, and standardize interactions with the GPT-4 model for all images. This prompt component contained both task-specific instructions and general information about the dataset, ensuring consistency in the model's outputs. Specifically, the prompt provided contextual details about the images that are relevant to the domain-specific diagnosis, including magnification, staining method, and anatomical features that apply to all samples. The prompt requests the model to “role-play” as an expert in morphological analyses to enhance the precision of its responses. Furthermore, the model is instructed to classify the images based on characteristics visible in the images belonging to each class. Lastly, specific instructions are provided to ensure the model generates output in a consistent, programmatically parsable format. This system prompt not only improves the model's performance but also ensures the application of consistent criteria to all images in the dataset. FIG. 3 shows an illustration of the workflow for microscopy image classification using GPT-4o in the inventors' experiments.

The test set was divided into batches to comply with OpenAI request rate limits (though, of course, when a VLM is run as a frozen, enterprise-managed model, rate limits need not apply, and images can be provided in batches, all at once, one at a time, etc. In the inventors' experiments, each API request included the system prompt, the prompt set, and the test batch, in that order. The model output was parsed into a JSON file for further analysis.

Experimental Results. The dataset for this work consists of 2-D microscopy images of histologically stained 3-D structures in tissue sections through the cerebellum of 18 mice brains (9 Lurcher mutation, 9 wild-type control group). The classification task involves distinguishing Lurcher mutant mice from the wild type. All the images are captured at low magnification (10×) and stained with cresyl violet, a generic stain for all brain cells. Images from a random subset of 6 mice (3 Lurcher and 3 control) were used for prompting the GPT-4o model while the other 12 mice (6 Lurcher mutation, 6 wild-type controls) were used for testing. The test set contained a total of 1471 images.

Table 1, below, shows classification performance of the inventors' approach on test studies. The second column from the right shows the number of images from each animal classified by the model as Lurcher or wild-type, respectively (e.g., 48/2 means 48 images were predicted as Lurcher and 2 as wild-type). The predicted class for each mouse is deter-mined by majority voting where the final class reflects the higher number of predicted images for that mouse.

TABLE 1

| Test | Ground | # of Predictions | Predicted |
|---|---|---|---|
| Animal ID | Truth Class | (Lurcher/Wild) | Class |
| 5917 | Lurcher | 48/2 | Lurcher |
| 6323 | Lurcher | 39/6 | Lurcher |
| 6350 | Lurcher | 24/50 | Wild |
| 6480 | Lurcher | 50/0 | Lurcher |
| 6481 | Lurcher | 38/0 | Lurcher |
| 6509 | Lurcher | 61/0 | Lurcher |
| 5973 | Wild | 1/171 | Wild |
| 6132 | Wild | 0/202 | Wild |
| 6134 | Wild | 4/171 | Wild |
| 6349 | Wild | 5/251 | Wild |
| 6353 | Wild | 2/135 | Wild |
| 6483 | Wild | 16/195 | Wild |

As a basis of comparison, the inventors utilized a microscopic image classification snapshot ensemble based on a CNN architecture, reported in the inventors' prior work. This baseline model serves as a comparison point for evaluating the relative efficiency and ground truth preparation time in this study.

The classification results of our approach on the 12 test animals are presented in Table 1. A total of 11 of 12 mice were predicted correctly with a significant margin of correct predictions for all 11 mice, resulting in an overall accuracy of 92%.

Tables 2 and 3, below, provide a comparison between the accuracy and ground-truth annotation time for two studies performed by the inventors' using APT-based software approaches against their respective baseline methods. Both methods (referenced as APT and APT-USF) demonstrate high classification accuracy, with APT achieving 91% accuracy (Table 2) and APT-USF achieving 92% accuracy (Table 3). The APT approach in the first study demonstrated an 86% reduction in annotation time compared to the baseline (Table 2), whereas the APT approach from the second study achieved a 96% reduction in annotation time (Table 3), despite being evaluated on a different dataset with more challenging characteristics.

Depicted below, Table 2 compares accuracy and ground-truth annotation time (in minutes). The ‘Improvement (%)’ column denotes the percentage reduction in time taken by the APT-based method compared to the baseline method. Table 3 compares accuracy and estimated ground-truth annotation time (in minutes) between the APT-based method of the inventors' second study versus the baseline method.

TABLE 2

| Method | Accuracy (%) | Time | Improvement (%) |
|---|---|---|---|
| APT | 91 | 92 | 86 |
| Baseline | 91 | 660 | — |

TABLE 3

| Method | Accuracy (%) | Time* | Improvement (%) |
|---|---|---|---|
| APT-USF | 92 | 45 | 96 |
| Baseline | N/A | 1080 | — |

This data demonstrates that methods utilizing the approaches and techniques disclosed herein can efficiently and accurately classify complex images in a nuanced diagnostic task that is highly domain-specific. In the inventors' studies, data from two diverse microscopy datasets with different characteristics (microglial cells in the hippocampus and diverse cells in the cerebellum) captured at different magnifications (10× and 20×) were tested, and consistency in improved results was achieved. These approaches have thus been proven to generalize across diverse data while maintaining an average accuracy of 92% and improving efficiency (annotation time savings) by an average of 91% compared to the baseline.

Moreover, beyond simply improved efficiency, the approaches herein also provide a further benefit: the generation of output explanations for each image in a style, format, wording, and scope matching the human expert review's input as well as established domain-specific knowledge and protocols. These explanations have strong value to researchers, medical lab staff, radiologists, pathologists, clinicians, insurers, healthcare providing organizations, and others as they provide a human-confirmable rationale (in a ‘native’ to the domain format) that associates specific features in the image with the domain-relevant diagnostic criteria.

### Practical Applications and Contemplated Implementations

Specific examples of contemplated use cases, implementation configurations, and embodiments will now be described. These descriptions are not meant as a closed set of how the advantages of the present disclosure can be used; rather they are meant as examples or guideposts to further illustrate the breadth of possible configurations and contemplated uses.

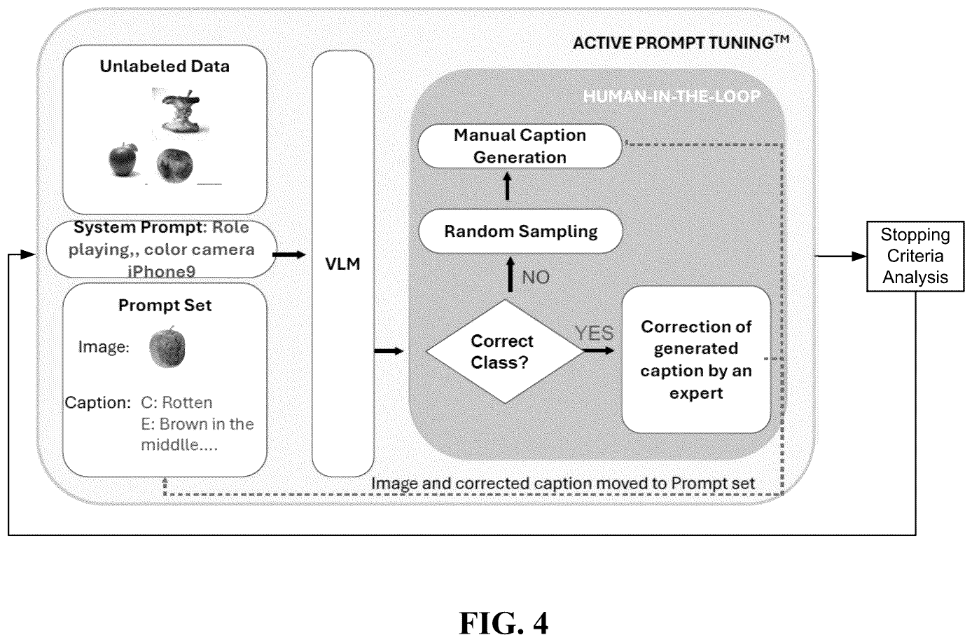

FIG. 4 is a conceptual flowchart depicting an implementation of a fruit quality diagnostic task, implemented via a multi-part software framework that manages interaction with a VLM and controls iterative refinement of such interaction. In this embodiment, an unlabeled active set includes images of fruit of various conditions. As shown, the images can be of different types of fruit and/or different varieties of fruit. Importantly, the diagnostic task is not merely to generate literal descriptions of extracted visual features of the images of fruit (e.g., shiny, red, has stem, has brown spots, etc.), but to diagnose and categorize them according to a predefined set of conditions (e.g., fruit grades or categorizations) which can be tailored to domain-specific terminology, buyer-specific criteria, or a combination thereof. As shown, the current candidate image is diagnosed as “rotten” meaning it is not usable for any fresh-market or processing purposes. A human expert has initially generated this diagnosis and associated explanation text (or at least corrected and refined it). The system prompt defines the role of the VLM, the source of the image (e.g., a field inspector's iPhone 9), image attributes (e.g., color), as well as the possible diagnostic categorizations (e.g., Organic Top Tier, Rotten, Second Tier, Processing-only, etc.) and explanations of the visual features that would be associated with each categorization. As described above, a human-in-the-loop active prompt tuning protocol is performed to fine-tune the instruction set to be provided to the VLM. Thus, in this example, a human expert may acquire images of many apples at once on-location via their iPhone, and select a diagnostic task definition and profile that is pre-stored for the type of apples being inspected or refine a new one via APT, then upload the new images to a cloud resource (e.g., device 202). The cloud resource may use computer vision techniques like object detection, edge detection, etc. to crop images of individual apples from the iPhone images, and process them via specific instructions to the VLM, then return predicted diagnoses with corresponding cropped individual apple images for the user to confirm. In some embodiments, summary information may also be provided indicating the number of apples in each category and general rationales for each type of categorization.

In one embodiment, a system is provided that comprises a library of predetermined instruction supplementation profiles for image diagnostic tasks. The profiles may comprise a diagnostic task definition, a plurality of Prompt Set examples, and a System Prompt, and each profile may be specific to a given classification or diagnostic task (e.g., determining presence of nodules, lesions, etc. within a specific region of interest in a class of medical images). Thus, a radiology laboratory or clinic deploying the system may automatically associate an instruction profile with a given scan protocol, so that the resulting images are automatically analyzed and preliminary diagnostic labels are provided in a review set to the radiologist. Where a new scan type or a new scan purpose is requested, the system may guide a radiologist through the methods described above (e.g., with respect to FIG. 1) for developing or refining a new profile.

For some repeatable diagnostic tasks, diagnostic task profiles may be obtained via a selection tool provided through a user interface. For example, in a dermatology diagnostic setting, a set of predetermined domain-specific and diagnostic-specific options may be available to a user to assess different types of skin lesions (e.g., pigmented lesion assessments, non-pigmented lesion assessments, inflammatory or infection-based lesion assessments, etc.). In such a situation, a software platform may be implemented as a diagnostic support tool, offering a dermatologist or support staff the ability to first determine a potential diagnosis or set of diagnoses, and then select one or more corresponding diagnostic profiles from a previously-generated library of profiles or elect to develop a new profile. In further embodiments, a new dermatologist may create their own profile by refining the labels and unstructured visual descriptions of a Prompt Set, or add/curate expert domain resources relied upon for System Prompts, so that the diagnostic support tools more closely align with the dermatologist's practice. In yet further alternatives, once a dermatologist has refined and approved profiles for image analysis, the dermatologist can adjust settings so that only certain future images/studies are presented for their review, while others (e.g., where a high accuracy rate and consistent input images are customary) can be presented to staff for only periodic confirmations.

In one example embodiment, the system described herein is integrated into a radiology software review platform configured to analyze computed tomography (CT) scans of a given region for the detection and classification of objects of interest like pulmonary nodules. In this implementation, the system (e.g., including processing and software resources like device 202) is embedded within a commercial DICOM and/or PACS (Picture Archiving and Communication System) platform used by radiologists for diagnostic interpretation. When a new CT scan is opened within the platform, an image preprocessing application/routine may automatically extract or obtain relevant metadata (e.g., scan protocol, anatomical region, slice thickness) and identify the diagnostic task as a specific type (e.g., “pulmonary nodule detection and classification”). Based on this task identification, the system retrieves a corresponding Prompt Set and System Prompt from the private prompt set library. The Prompt Set includes curated image-caption pairs representing a range of annotated nodules (e.g., benign, malignant, indeterminate) and associated morphological descriptors. The System Prompt provides task-specific instructions for the VLM that match the identified task, such as describing a task-appropriate role (radiologist experienced in review of CT scans for pulmonary diagnosis), providing accepted visual criteria for diagnoses, emphasizing human expert confirmation when appropriate, defining output types, and including uncertainty estimates in the output.

The preprocessed CT image slices may then be then passed to the frozen VLM with the System Prompt and Prompt Set corresponding to the study type. The VLM may then generate structured outputs according to the System Prompt, including bounding boxes, classification labels, and explanatory captions. These outputs are then validated through a subsequent software pipeline, including an output formatting module to ensure that they conform to the expected schema of the host PACS platform and that the labels used are consistent with the diagnostic vocabulary and taxonomy for thoracic imaging. The validated outputs may then be presented as overlays within the radiologist's viewer, with optional links to the Prompt Set examples that most closely resemble the images of the current patient's study. In some embodiments, an input monitoring module may detect a new pattern in the input images (e.g., a new distribution, shape, size, etc. of nodule or lesion appearances) and suggest that the radiologist conduct a profile refinement to update or branch the Prompt Set. The entire automated diagnostic process may occur locally within the PACS environment or via a secure connection to a remote instance of device 202.

In another example embodiment, a system is configured to remotely monitor and/or analyze video recordings of moving parts in a complex device, factory, or other installation of industrial machinery, to diagnose mechanical anomalies, maintenance needs, broken parts, etc. based on motion patterns. In this implementation, an automatic, autonomous monitoring and diagnostic application is deployed within a factory automation system or maintenance analytics platform. The input comprises a video sequence captured by a stationary camera positioned to monitor a moving part, such as a robotic arm, products on an assembly line, rotating shaft assembly of a turbine or pump, etc. The preprocessing module may be programed to utilize known computer vision techniques to trigger recording of a time series of frames from the camera in certain cycles or operations, or simply on a periodic regular basis. A segment of video frames can then be presented as an input for a predefined diagnostic task like “rotational anomaly detection.” Based on this task, the system retrieves a corresponding Prompt Set and System Prompt from the manufacturer's (or local) file storage. Each Prompt Set entry includes annotated video clips of the same or similar machinery exhibiting known issues such as shaft misalignment, unbalanced rotation, or bearing degradation, along with captions describing the observed motion irregularities and their likely causes.

When a new diagnostic profile is being made, the user interface (e.g., 208 as described above) can allow a technician or engineer (either associated with a manufacturer of the equipment or with the facility in which the equipment is installed or used) to tag specific regions of interest—such as the shaft, coupling, or housing—or to landmark key rotational phases in a video segment. The frozen VLM may then process a sequence of frames and generate outputs that include motion classification labels (e.g., “eccentric rotation,” “vibration anomaly”), temporal annotations, and explanatory captions. The output formatting module ensures that these results are compatible with the host maintenance platform and may trigger alerts or generate structured reports for further review. In some embodiments, the input monitoring module may detect a shift in operational conditions (e.g., increased vibration amplitude due to heavier fluids being pumped, frame blur due to lighting changes or assembly line speeds, etc.) and initiate a refinement process to update the Prompt Set. The system may operate locally on an edge device near the machinery or connect to a centralized instance of device 202 via a network 220, to facilitate privacy, proprietary or security concerns, as well as latency and bandwidth constraints.

As used in this specification and the claims, the singular forms “a,” “an,” and “the” include plural forms unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or “B” or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use an aspect of a described embodiment or feature contained in the same, or a definitive decision to use a specific skill regarding a described embodiment or feature contained in the same. In this latter context, the modal verb “may” has the same meaning and connotation as the auxiliary verb “can.”

In the foregoing specification, implementations of the disclosure have been described with reference to specific example implementations thereof. It will be evident that various modifications may be made thereto without departing from the broader spirit and scope of implementations of the disclosure as set forth in the following claims. The specification and drawings are, accordingly, to be regarded in an illustrative sense rather than a restrictive sense.

1. A method for providing active prompt tuned automated microscopy image analysis comprising:
receiving criteria information for a given class of microscopy study, the criteria information comprising: a set of possible slide classification labels, descriptions of a set of predefined visual features of anatomy anticipated to be found in microscopy slide images for each of the slide classification labels, and slide image preparation information describing:
a sample acquisition method used to prepare biological samples appearing in the slides;
a stain method used to stain structures within the samples; and
a magnification used to acquire images of the samples;
receiving a set of unclassified slide images prepared using the sample acquisition method, the stain method, and the magnification;
creating an Active Set of unclassified images by random sampling of the set of unclassified images;
generating a System Prompt based on the criteria information and input/output characteristics of a diagnostic review platform for microscopy images;
presenting an Initial Prompt subset of the Active Set of images to a human reviewer, and requiring the human reviewer to select one of the image classification labels and provide an unstructured feature explanation, for each image of the Initial Prompt subset;
providing images of the Active Set as input to a vision-language model (VLM) with an instruction comprising the System Prompt and a Prompt Set comprising the Initial Prompt subset;
in an iterative fashion, expanding the Prompt Set by performing (i)-(iii) until the Prompt Set contains a threshold number of image-caption pairs exhibiting each label of the slide classification labels and each feature of the set of visual features:
displaying to the human reviewer candidate image-caption pairs comprising images of the Active Set together with associated predicted labels and predicted unstructured feature explanations derived from VLM outputs;
requiring the human reviewer to review the image-caption pairs and confirm, reject, or edit them; and
for each image-caption pair approved or edited by the human reviewer, adding them as an entry to the Prompt Set;
generating a diagnostic profile specific to the given class of microscopy study based on the System Prompt and Prompt Set; and
storing the diagnostic profile in a memory associated with the diagnostic review platform for use in automatically generating image classifications and supporting feature explanations for future patient slide images of the given class of microscopy study.

2. The method of claim 1 further comprising presenting a user interface of the diagnostic review platform to allow a user to select among stored diagnostic profiles for a plurality of classes of microscopy study.

3. The method of claim 1 further comprising monitoring patient slide images reviewed via the diagnostic review platform for the given class of microscopy study to determine changes in average image attributes relating to the set of visual features, and if such a change is detected, generating a second Active Set from among such patient slide images and requiring a human reviewer to iteratively supplement the Prompt Set.

4. The method of claim 1, wherein image-caption pairs are not admitted to the Prompt Set until having been approved more than once by a human reviewer.

5. The method of claim 1, wherein slide images of the Active Set that are rejected five (5) times during iterative expansion of the Prompt Set are removed from the Active Set.

6. The method of claim 1, wherein the unclassified slide images comprise stacks of image slices of three-dimensional biological samples, and further wherein the stacks are provided as inputs to the VLM as groups, and the System Prompt instructs the VLM on the spatial relationship of the images and instructs the VLM to generate one predicted classification label and one unstructured explanation for each group.
